(19) United States
(12) Patent Application Publication
(10) Pub. No.: US 2003/0207312 A1
(43) Pub. Date: Nov. 6, 2003

(54) Gene monitoring and gene identification using cDNA arrays

(75) Inventor: Joseph A. Sorge, Wilson, WY (US)

Correspondence Address: PALMER & DODGE, LLP, KATHLEEN M. WILLIAMS / STR, 111 HUNTINGTON AVENUE, BOSTON, MA, 02199, US

(73) Assignee: Stratagene

(21) Appl. No.: 10/447,806

(22) Filed: May 29, 2003

Related U.S. Application Data
(63) Continuation of application No. 09/709,945, filed on Nov. 10, 2000, now abandoned.

(51) Int. Cl. C12Q 1/68; C12M 1/34

(52) U.S. Cl. 435/6; 435/287.2

(57) ABSTRACT

cDNA microarrays are provided for increasing the accuracy and reliability of expression profiling techniques and for identifying new genes. An array comprises a plurality of nucleic acid members, each member having a unique position and stably associated with a solid support. Each nucleic acid member comprises a noncoding sequence present at either the 3′-end or the 5′-end of an RNA transcript (e.g., such as an untranslated region or UTR). In one embodiment, each nucleic acid member is less than 1000 nucleotides. In another embodiment, each nucleic acid member is less than 600 nucleotides. In a further embodiment of the invention, each nucleic acid member comprises substantially noncoding sequences.

### PRIORITY

This application is a continuation of application Ser. No.09/709,945, filed Nov. 10, 2000. The entire teachings of the above application is incorporated herein by reference.

### FIELD OF THE INVENTION

The invention relates to a cDNA array for monitoring gene expression and for identifying novel genes.

### BACKGROUND OF THE INVENTION

When and where a gene is expressed provides clues as to its biological role. The large and ever expanding databases of complementary DNA (cDNA) sequences, Expressed Sequence Tag (EST) sequences, as well as entire genome sequences from many organisms, present the opportunity to define patterns of gene expression representative of an entire cell, tissue, or organism, enabling an expression profile to be created for that cell, tissue, or organism, in both healthy and pathological states. An understanding of the biological relevance of complex gene expression patterns requires the implementation of sophisticated methods for gene expression analysis and gene discovery.

Over the last five years, array-based methods for high-throughput monitoring of gene expression have been described which permit the evaluation of multiple genes simultaneously. These methods involve using fragments of genes or cDNAs arrayed at a plurality of positions on a substrate (e.g., arrays) to create gene-specific hybridization targets for a population of RNA molecules obtained from a cell, tissue, or organism sample. RNA molecules which hybridize to the array, and those which do not, provide information regarding the expression profile of the sample being tested. cDNA arrays, or arrays which include only transcribed sequences, offer advantages over gene arrays in that only targets which are actually expressed are presented to a sample, maximizing the information which is obtainable from the hybridization signals observed.

Despite the potential offered for expression profiling using cDNA arrays, cDNA arrays known in the art suffer from several drawbacks. For instance, in order to obtain an accurate expression profile of an RNA sample, it is critical that a hybridization signal obtained at a given position on the array correspond to a single cDNA molecule; in other words, each cDNA arrayed on the substrate should have a unique position on the array and that position should be known.

However, in practice, the arraying of CDNA clones often proves to be problematic. Most cDNA microarrays are created by spotting small amounts of PCR products obtained from plasmid cDNA templates onto glass microscope slides. Such PCR products are typically generated using two vector-specific primers that anneal to priming sites flanking the cDNA insert. There have been many reports that the DNA spotted onto such arrays is often a mixture of more than one clone or is an incorrect clone. The authenticity of a given clone that has been spotted onto an array can therefore be questionable, as neither its position nor its uniqueness are known with certainty.

An additional problem arises where a cDNA is unique in terms of its overall sequence, but shares similar or identical subsequences with other cDNAs on the microarray. As a consequence, multiple hybridization targets can be created under hybridization conditions typically used in screening where only one real target exists. This problem is compounded in ordered microarrays which provide cDNAs grouped into families based on regions of sequence similarity in coding sequences (e.g., multiple similar targets are grouped within the same location on the array). In addition to the ability of coding regions to cross hybridize, 3′ untranslated regions sometimes contain repeat elements, such as Alu sequences, which can cross hybridize, making any correlation between a hybridization signal and the expression of a specific gene suspect.

In view of the difficulties associated with analysis of gene expression by cDNA arrays, there continues to be interest in the development of cDNA arrays which increase the probability of identifying the expression of specific genes. There is also a need in the art for improved cDNA array methodology that will increase the opportunity for novel gene identification.

### SUMMARY OF THE INVENTION

The invention relates to a cDNA array for increasing the accuracy and reliability of expression profiling techniques and for identifying new genes. In one embodiment of the invention, an array is provided comprising a plurality of nucleic acid members, each member having a unique position and stably associated with a solid support. Each nucleic acid member comprises a noncoding sequence present at either the 3′-end or the 5′-end of an RNA transcript (e.g., such as an untranslated region or UTR). In one embodiment, each nucleic acid member is less than 1000 nucleotides. In another embodiment, each nucleic acid member is less than 600 nucleotides. In a further embodiment, each nucleic acid member comprises a noncoding sequence present at either the 3′-end or the 5′-end of an RNA transcript which ranges from 20 nucleotides to 700 nucleotides. In a further embodiment of the invention, each nucleic acid member comprises substantially noncoding sequences.

In one embodiment of the invention, each nucleic acid sequence has a unique and known position on the substrate with which it is stably associated. In another embodiment, nucleic acid members comprise both known and unknown sequences (with respect to publicly available databases) and each nucleic acid member is identified as a known or unknown sequence prior to being stably associated with the substrate. In a further embodiment of the invention, information relating to whether a nucleic acid member is known or unknown is stored within the memory of a computer or a computer program product along with information relating to the position of the nucleic acid member on the substrate of the array.

In another embodiment of the invention, a composition is provided comprising a plurality of at least two different nucleic acid members, each nucleic acid member comprising a non-coding sequence present at either a 3′-end or 5′-end of an RNA transcript. In one embodiment of the invention, each of said nucleic acid members is less than 1000 nucleotides. In another embodiment of the invention, each nucleic acid member is less than 600 nucleotides. In a further embodiment of the invention, each nucleic acid member comprises substantially noncoding sequences.

In another embodiment, the invention provides a method of producing a cDNA array. The method comprises selecting a cDNA sequence (e.g., a plasmid clone comprising a cDNA sequence) at random from a population of cDNA sequences (e.g., a cDNA library). The sequence of at least a portion of the 3′ end of the cDNA is determined to identify a complementary sequence suitable for use as an amplification primer (e.g., a 3′-end PCR primer). Amplification is performed by providing the 3′-end primer, a polymerase, nucleotides, and an amplification buffer, and the primer is extended by the polymerase to generate a nucleic acid member which comprises the non-coding sequence present at the 3′-end of an RNA transcript corresponding to the cDNA.

In a further embodiment of the invention, the cDNA comprises at least one constant sequence (e.g., vector sequences or an adapter sequence) contiguous with the 5′-end of the cDNA molecule, and present in each cDNA molecule in the population. A primer corresponding to the constant sequence of the molecule is included in the amplification reaction to generate an amplified sequence or nucleic acid member which comprises the non-coding sequence present at the 3′-end of an RNA transcript corresponding to the CDNA and at least a portion of the constant sequence. In one embodiment of the invention, the cDNA sequence contains substantially non-coding sequences and excludes repeat elements (e.g., Alu elements). In another embodiment, the nucleic acid member does not contain vector sequences or adapter sequences contiguous with, at least its 3′-end.

In a further embodiment of the invention, the sequence information obtained from at least a portion of the 3′-end of the CDNA is compared to sequence information in a public database, and the CDNA is identified as a known sequence if there is substantial identity between the sequence of at least a portion of the 3′-end and a sequence in the database. If there is no substantial identity, the cDNA is identified as an unknown sequence, and sequence information relating to the cDNA is stored within the memory of a computer or a computer program product. In one embodiment, at least 2% of the population of CDNA molecules used to generate the CDNA array, does not contain significant sequence identity to a nucleic acid sequence in a public database. In other embodiments, at least 5%, 10%, 15% or 20% of the population of cDNA molecules used to generate the cDNA array, does not contain significant sequence identity to a nucleic acid sequence in a public database.

The nucleic acid member is stably associated with a substrate at a unique position on the substrate, and additional randomly selected cDNA sequences are sequenced to identify complementary sequences suitable for use as amplification primers and to generate additional nucleic acid members. Each nucleic acid member is stably associated with a different unique position on the substrate, generating an array of cDNA sequences. In one embodiment of the invention, each nucleic acid member on the array is less than 600 nucleotides. In another embodiment of the invention, each nucleic acid member comprises a non-coding region ranging from 20-700 nucleotides. In still another embodiment of the invention, each nucleic acid member contains substantially noncoding sequences.

In another embodiment, a cDNA array is produced in which nucleic acid members comprise a non-coding sequence present at the 5′-end of an RNA transcript. The method comprises selecting a cDNA sequence (e.g., a plasmid clone comprising a cDNA sequence) at random from a population of CDNA sequences (e.g., a cDNA library). The sequence of at least a portion of the 5′-end of the cDNA is determined to identify a complementary sequence suitable for use as an amplification primer (e.g., a 5′-end PCR primer). Amplification is performed by providing the 5′-end PCR primer, a polymerase, nucleotides, and an amplification buffer, and the primer is extended by the polymerase to generate a nucleic acid member which comprises the non-coding sequence present at the 5′-end of an RNA transcript corresponding to the cDNA. In a further embodiment of the invention, the CDNA comprises at least one constant sequence (e.g., vector sequences or an adapter sequence) contiguous with the 3′-end of the cDNA molecule and present in all of the cDNAs in the population. A primer corresponding to the constant sequence end of the molecule is included in the amplification reaction to generate an amplified sequence or nucleic acid member which comprises the non-coding sequence present at the 5′-end of an RNA transcript corresponding to the cDNA and at least a portion of the constant sequence. In one embodiment of the invention, the cDNA sequence contains substantially non-coding sequences and excludes repeat elements (e.g., Alu elements). In another embodiment, the nucleic acid member does not contain vector sequences or adapter sequences at the 5′-end of the nucleic acid member.

In a further embodiment of the invention, the sequence information obtained from at least a portion of the 5′-end of the cDNA is compared to sequence information in a public database, and the cDNA is identified as a known sequence if there is substantial identity between the sequence of at least a portion of the 5′-end and a sequence in the database. If there is no substantial identity, the CDNA is identified as an unknown sequence, and sequence information relating to the cDNA is stored within the memory of a computer or a computer program product. In one embodiment, at least 2% of the population of cDNA molecules used to generate the cDNA array, does not contain significant sequence identity to a nucleic acid sequence in a public database. In other embodiments, at least 5%, 10%, 15% or 20% of the population of cDNA molecules used to generate the CDNA array, does not contain significant sequence identity to a nucleic acid sequence in a public database. Preferably, the CDNA library comprises clones of human CDNA sequences; however, in other embodiments of the invention, the cDNA library comprises clones of non-human species, including, but not limited to mice, rats, frogs, fruitflies, nematodes, and plant cDNA sequences.

The nucleic acid member comprising the non-coding sequence present at the 5′-end of an RNA transcript is stably associated with a substrate at a unique position on the substrate. The steps of the method are repeated, either sequentially or simultaneously, and additional randomly selected cDNA sequences are selected and sequenced to identify complementary sequences suitable for use as amplification primers (5′-end primers) to generate additional nucleic acid members. Each nucleic acid member is then stably associated with a different unique position on the substrate, generating an array of CDNA sequences. In one embodiment of the invention, each nucleic acid member on the array is less than 1000 nucleotides. In another embodiment of the invention, each nucleic acid member comprises a non-coding region ranging from 20-700 nucleotides. In another embodiment of the invention, each nucleic acid member contains substantially noncoding sequences.

In a further embodiment, the cDNA sequences comprising either 5′-end or 3′-end noncoding sequences comprise human sequences. In still a further embodiment, the nucleic acid members comprise sequences from two or more tissues (e.g., human tissues). In one embodiment of this aspect of the invention, at least 2% of the population of cDNA molecules used to generate the cDNA array, does not contain significant sequence identity to a nucleic acid sequence in a public database. In other embodiments, at least 5%, 10%, 15% or 20% of the population of CDNA molecules used to generate the cDNA array, does not contain significant sequence identity to a nucleic acid sequence in a public database.

The invention further provides a method of analyzing the expression of one or more genes. The method comprises hybridizing a sample to an array comprising a plurality of nucleic acid members, each member having a unique position and stably associated with a solid substrate and each nucleic acid member comprising a non-coding sequence present at either a 3′-end or 5′-end of an RNA transcript. In one embodiment, each nucleic acid member is less than 1000 nucleotides. In another embodiment, each nucleic acid member is less than 600 nucleotides. In a further embodiment, each nucleic acid member comprises at least 20-700 nucleotides of a non-coding sequence found in an RNA transcript. In one embodiment of the invention, none of the nucleic acid members on the array comprises vector sequences contiguous with the noncoding sequences. In still a further embodiment of the invention, each nucleic acid member contains substantially noncoding sequences.

By determining whether any expressed target nucleic acid sequence (e.g., mRNA) within a sample hybridizes to the array, data relating to the expression of the target nucleic acid sequence in the sample is obtained. In one embodiment of the invention, the data comprises the amount of target nucleic acid sequence expressed in a sample. In another embodiment of the invention, the data comprises the identity of the nucleic acid member to which the target nucleic acid sequence hybridizes (e.g., a known or unknown sequence). In another embodiment of the invention, a nucleic acid member comprising an unknown sequence which has hybridized to a target nucleic acid sequence is sequenced. In a further embodiment of the invention, the sequence of the known or unknown sequence is entered into the memory of a computer or a computer program product and the sequence is identified as a known sequence and information about its expression pattern is entered into the memory of the computer or computer program product.

In still a further embodiment of an invention, an expression profile is generated comprising data related to the expression of a gene or group of genes in a biological system (e.g., a cell, group of cells, tissue, group of tissues, organ, or organism), in healthy and pathological states (where the biological system is subject to genetic alterations and/or environmental disturbances) using the arrays of the invention. In another embodiment, the biological relevance of a previously unknown or uncharacterized gene is determined by determining the expression profile of this gene in a biological system. In still another embodiment, the expression profile of a previously unknown or uncharacterized gene is compared to the expression profile of other genes. In still a further embodiment, compared profiles are used to identify interactions between genes.

### BRIEF DESCRIPTION OF THE DRAWINGS

The objects and features of the invention can be better understood with reference to the following detailed description and accompanying drawings.

FIG. 1A is a schematic illustration of production of a cDNA array comprising noncoding sequences present at the 3′-end of an RNA transcript of one embodiment of the invention. FIG. 1B is a schematic illustration of production of a cDNA array comprising noncoding sequences present at the 5′-end of an RNA transcript of one embodiment of the invention.

FIG. 2 is a schematic diagram of a method of computing the percent alignable sequences useful for classifying sequences as known or unknown.

### DESCRIPTION

The invention provides cDNA arrays comprising a plurality of nucleic acid members, each nucleic acid member having a unique position and stably associated with a substrate. Each nucleic acid member comprises noncoding sequences present at either the 3′-end or the 5′-end of an RNA transcript (e.g., such as an untranslated region or UTR) and in one embodiment, none of the nucleic acid members on the array comprises vector sequences or adapter sequences contiguous with the non-coding sequence. In another embodiment of the invention, each nucleic acid member comprises at least 20 to 700 nucleotides of the noncoding sequence of an RNA transcript. In still another embodiment of the invention, each nucleic acid member comprises substantially non-coding sequences. Methods and compositions for generating the arrays and methods of using the arrays to monitor gene expression or identify novel genes are also proyided.

Definitions

In order to more clearly and concisely describe and point out the subject matter of the claimed invention, the following definitions are provided for specific terms which are used in the following written description and the appended claims.

As used herein, “3′-end of an RNA transcript” refers to at least 8 and less than 600 contiguous nucleotides of the end of an mRNA that is immediately adjacent to the polyA tail and extends toward the 5′-end of the mRNA. The “3′-end of an RNA transcript” includes 3′ untranslated sequences or noncoding sequences, and may or may not contain coding sequence from the 3′ portion of the coding region of an mRNA. Preferably, the “3′-end of an mRNA” includes primarily noncoding sequences (90%-l100% of the 3′ end is untranslated or noncoding sequence), and thus includes only a relatively short portion that is translated, or is part of a coding region.

As used herein, “5′-end of an RNA transcript” refers to at least 8 and less than 1000 contiguous nucleotides of the end of a full length mRNA that includes and is adjacent to the most 5′ nucleotide of a full length mRNA, and extends toward the 3′-end of the mRNA (e.g., toward the polyA tail). The “5′-end of an RNA transcript” includes 5′ untranslated sequences and may or may not contain coding sequence from the 5′ portion of the coding region of a mRNA. Preferably, the “5′-end of an RNA transcript” includes primarily noncoding sequences (90%-100% of the 5′ end is untranslated or noncoding sequence), and thus includes only a relatively short portion that is translated, or is part of a coding region.

As used herein, “a sequence at the 5′ end” or “at the 3′-end” of an RNA transcript is a nucleic acid sequence from the 5′- or 3′-end of an mRNA sequence which is less than 50% of the transcript and which includes the 5′ most nucleotide or the 3′ most nucleotide adjacent to the polyA tail, respectively.

As used herein, a nucleic acid sequence which “contains substantially noncoding sequences” refers to a nucleic acid sequence which encodes less than 50% of a full length protein.

As used herein, the term “coding region” refers to the portion of a gene, mRNA or cDNA that encodes the amino acids of a polypeptide encoded by the gene. The 5′ portion of the coding region corresponds to the amino-terminal portion of the encoded polypeptide and is less than, or equal to, 50% of the entire coding region, while the 3′ portion of the coding region corresponds to the carboxy-terminal portion of the encoded polypeptide and is less than, or equal to 50% of the entire coding region.

As used herein a “sequence suitable for use as an amplification primer” is one which has sequence properties which permit it to specifically hybridize under amplifying conditions to a sequence to be amplified. Sequencing primers are generally from 5 nucleotides in length to 100 nucleotides in length and are preferably from 6 to 50 nucleotides in length.

As used herein, “amplifying conditions” are conditions under which a polymerase will extend a primer sequence which is hybridized to a sequence to be amplified to produce a sequence complementary to the sequence to be amplified.

As used herein, a “nucleic acid member” comprises either a single stranded or double stranded nucleic acid which comprises a noncoding sequence present at either the 3′-end or the 5′-end of an RNA transcript. As defined herein, a “single nucleic acid member” comprises one or more nucleic acid molecules which are identical in sequence to each other. A nucleic acid member which is “not identical in sequence” to another nucleic acid member will contain at least a single nucleotide difference, and may contain 10, 20, 50, 100, 200 or more nucleotide sequence differences, with respect to an alignment of the sequences that provides the maximum amount of homology; if no such alignment exists, then with respect to the nucleotide alignment starting at the 3′ or 5′ ends of the sequences. Sequence differences also may be determined solely with respect to the noncoding sequences of the members.

As used herein, a “nucleic acid molecule” is a molecule which can bind via Watson Crick bonds to another nucleic acid molecule, and can include nucleotides naturally present in a cell or modified nucleotides.

As used herein, a “modified nucleotide” is a nucleotide which comprises an altered base and/or altered sugar and/or altered intemucleotide linkage but which can still incorporate into a nucleic acid molecule via an intemucleotide linkage and form at least Watson Crick bonds with another nucleotide.

As used herein, “altered” refers to a chemical group which is not present in a naturally occurring nucleotide.

As used herein, an “array” comprises a plurality of nucleic acid members stably associated with a substrate. The term “array” is used interchangeably with the term “microarray,” however, the term “microarray” is used to define an array which has the additional property of being viewable microscopically.

As used herein, “viewable microscopically” refers to an object which can be placed on the stage of a dissecting or compound microscope and comprises at least a portion which can be viewed using an ocular of the microscope.

As, used herein, “stably associated” refers to an association with a position on a substrate that does not change under nucleic acid hybridization and washing conditions.

As used herein, “specific hybridization” refers to the binding, duplexing, or hybrization of a molecule only to a target nucleic acid sequence and not to other non-target nucleic acid molecules in a mixture of both target and non-target nucleic acid sequence.

As used herein, “cDNA” (complementary DNA) refers to a DNA sequence which is the exact complement of an mRNA sequence. A cDNA which “corresponds” to an mRNA sequence is a cDNA which is an exact complement of that mRNA sequence.

As used herein, a “position” refers to a site on a substrate that is distinguishable from any other site on the substrate either by eye or by an optical instrument. A “unique position” refers to a position which comprises a single nucleic acid member.

As used herein, an “unknown sequence” is a sequence not included in a public nucleic acid sequence database at the time the array was generated, either as a complete gene sequence, a partial gene sequence, a cDNA, or an expressed sequence tag (EST).

As used herein, a “vector sequence” is a sequence obtained from an extrachromosomal DNA which can replicate independently of chromosomal DNA, and includes plasmid, cosmid, phagemid, bacteriophage DNA, and the like.

As used herein, “substantially identical sequences” refers to a least two nucleic acid members which are at least 95% identical when aligned for maximum correspondence over a comparison window of 100 nucleotides, and preferably 50-600 nucleotides.

cDNA Arrays Comprising Noncoding Sequences

The invention relates to a cDNA array for increasing the accuracy and reliability of expression profiling techniques and for identifying new genes. In one embodiment of the invention, an array is provided comprising a plurality of nucleic acid members, each member having a unique position and stably associated with a solid substrate

Each nucleic acid member comprises a noncoding sequence present at either the 5′-end or the 3′-end of an RNA transcript (e.g., such as an untranslated region or UTR). The invention also provides for nucleic acid members comprising a noncoding sequence present at both the 5′-end and the 3′ -end of the RNA transcript. In one embodiment, each nucleic acid member is less than 1000 nucleotides. In another embodiment, each nucleic acid member is less than 600 nucleotides. In a further embodiment, a nucleic acid member comprising the noncoding sequence present at the 3′-end of an RNA transcript does not comprise vector sequences or adapter sequences contiguous with the noncoding sequence present at the 3′-end. In another embodiment, a nucleic acid member comprising the 5′-end of an RNA transcript does not comprise vector sequences or adapter sequences contiguous with the 5′-end. In a preferred embodiment of the invention, neither the 5′- nor the 3′-end of the nucleic acid member comprises vector sequences or adapter sequences. In a further embodiment, the size of the noncoding sequences range from 20 nucleotides to 700 nucleotides.

In one embodiment of the invention, a nucleic acid member comprises a sequence at the 5′-end of an RNA transcript and which is less than 50% of the length of the full length transcript. In one embodiment, the nucleic acid member is any of: 950 nucleotides, 900 nucleotides, 890 nucleotides, 850 nucleotides, 800 nucleotides, 750 nucleotides, 700 nucleotides, 650 nucleotides, 600 nucleotides, 590 nucleotides, 550 nucleotides, 500 nucleotides, 450 nucleotides, 400 nucleotides, 350 nucleotides, 300 nucleotides, 250 nucleotides, 200 nucleotides, 150 nucleotides, 100 nucleotides, 50 nucleotides, 20 nucleotides, 15 nucleotldcs, 10 nucleotides, or 8 nucleotides in length.

In another embodiment, a nucleic acid member comprises a sequence at the 3′ end of an RNA transcript and which is less than 50% of the length of the full length transcript. In one embodiment, the nucleic acid member is any of: 595 nucleotides, 590 nucleotides, 550 nucleotides, 500 nucleotides, 450 nucleotides, 400 nucleotides, 350 nucleotides, 300 nucleotides, 250 nucleotides, 200 nucleotides, 150 nucleotides, 100 nucleotides, 50 nucleotides, 20 nucleotides, 15 nucleotides, 10 nucleotides, and 8 nucleotides.

In one embodiment of the invention, each nucleic acid member contains substantially noncoding sequences and encodes less than 50% of a full length protein encoded by the RNA transcript which corresponds to the nucleic acid member. In another embodiment of the invention, the nucleic acid member encodes less than 45%, less than 40%, less than 30%, less than 20%, less than 10%, and less than 5% of the full length protein encoded by the RNA molecule. In a further embodiment of the invention, none of the nucleic acid members on the array comprise vector sequences contiguous with the noncoding sequence of the nucleic acid member.

In one embodiment, each position on the array comprises a nucleic acid member which is nonidentical (i.e., there is at least one nucleotide difference between each nucleic acid member, and preferably, there are 2, 3, 4, 5, 6, 10, 20, 50, 100, or more nucleotide differences) to nucleic acid members at any other position. In one embodiment of the invention, at least 50% of the positions on the substrate comprise nonidentical nucleic acid members. In another embodiment of the invention, 55%, 60%, 65%, 70%, 75%, 80% or 100% of the positions comprise nonidentical nucleic acid members.

In one embodiment of the invention, nucleic acid members comprise natural nucleotides (e.g., deoxyribonucleotides, or ribodeoxynucleotides). In another embodiment of the invention, at least one nucleic acid member comprises at least one modified nucleotide to enhance the resistance of the array to nucleases. In one embodiment, modified nucleotides can include one or more substitute internucleotide linkages, altered sugars, altered bases, or combinations thereof. In one embodiment of the invention, nucleotides are provided in which the P(O)O group is replaced by P(O)S (“thioate”), P(S)S (“dithioate”), P(O)NR2 (“amidate”), P(O)R, P(O)OR′, CO or CH2 (“formacetal”) or 3′-amine (—NH—CH2—CH2—), wherein each R or R′ is independently H or substituted or unsubstituted alkyl. Linkage groups can be attached to adjacent nucleotides through an —O— linkage or through an —N— or —S— linkage. Not all linkages in the nucleic acid member sequences are required to be identical. In further embodiments of the invention, the nucleotides comprise modified sugar groups, for example, comprising one or more of the hydroxyl groups replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group.

Methods of synthesizing modified nucleotides are well known, including, for example, the phosphotriester method described by Narang et al., 1979, Methods in Enzymology, 68:90, the phosphodiester method disclosed by Brown et al., 1979, Methods in Enzymology, 68:109, the diethylphosphoramidate method disclosed in Beaucage et al., 1981, Tetrahedron Letters, 22:1859, and the solid support method disclosed in U.S. Pat. No. 4,458,066, or by other chemical methods using either a commercial automated oligonucleotide synthesizer (which is commercially available) or VLSIPST technology, the entireties of which are incorporated by reference herein. Teachings regarding the synthesis of particular modified oligonucleotides may be found in the following U.S. patents, U.S. Pat. Nos. 5,138,045, 5,218,295, 5,218,105, 5,212,295, 5,378,825, 5,547,191, 5,459,255, 5,521,302, 5,539,082, 5,571,902, 5,578,718, 5,506,351, 5,587,470, 5,608,046, and 5,459,255, the entireties of which are incorporated herein by reference.

Substrates which are encompassed within the scope of the present invention comprise flexible and non-flexible substrates, porous and nonporous substrates which exhibit a low level of non-specific binding during hybridization events. Suitable substrates of the invention, include, but are not limited to, glass (e.g., sialated glass, Bioglass®); ceramics; polymers, including plastics, e.g. polytetrafluorethylene, polypropylene, polystyrene, polycarbonate, and blends thereof, and the like; metals, e.g., gold, platinum, and the like; nylon, both modified and unmodified; celluosic materials (e.g., nitrocellulose), cellulose acetate; poly (vinyl chloride); polyacrylamide; cross linked dextran; agarose; polyacrylate; polyethylene; polypropylene; poly (4-methylbutene); polymethacrylate; poly(ethylene terephthalate); nylon; poly(vinyl butyrate); and the like; and combinations thereof. In one embodiment of the invention, the substrate comprises a plurality of positively charged molecules on its surface.

Substrates can have any number of shapes, such as strip-shaped, planar, disc-shaped, bead-shaped, and the like. Nucleic acid members can be stably associated with a substrate by a variety of means well known in the art. Stable associations can be achieved by crosslinking (e.g., by ultraviolet irradiation, by heat, by mechanical or chemical bonding procedures, by using a vacuum system, or through a combination of techniques). In one embodiment of the invention, amino functionalities are attached to the 5-end of the nucleic acid member and linker groups are used to attach the amino group to the surface of an amine-reactive solid support (see, e.g., U.S. Pat. No. 6,077,674, the entirety of which is incorporated by reference herein).

Nucleic acid members can be stably associated with the substrate at different positions on the array using any convenient methodology, including manual techniques, e.g. by micro pipetting. Automated devices can also be used such as pin spotting devices, inkjet printers, and other automatic spotting or arraying devices (see, e.g., U.S. Pat. No. 5,770,151 and WO 95/35505, the entireties of which are incorporated by reference). Additional microfabrication technologies for stably associating nucleic acid members with a substrate include photolithography, micropatteming, light-directed chemical synthesis, laser stereochemical etching and microcontact printing (reviewed in Cheng et al., 1996, Mol. Diagn., 1:183-200).

In one embodiment of the invention, positions are separated from each other by locations on the substrate which are not stably associated with nucleic acid members. In one embodiment, the position to position distance on the substrate (i.e., from the midpoint of one position to the midpoint of an adjacent position) is from 5-1000 μm. Preferably, the position to position distance on the substrate is 100-500 μm. If nucleic acid members are stably associated with a substrate by the method of photolithography, the position to position distance on the substrate is preferably 5-50 μm. In one embodiment of the invention, each position on the substrate is distinguishable from any other position either visually or through the use of an optical instrument (e.g., such as a microscope, CCD array, photodiode array, and the like) or through the use of electrical instruments (e.g., devices communicating with capacitors or electrodes positioned under the substrate) which are capable of obtaining optical and electrical data, respectively, relating to substrate positions.

Positions can be any shape, and shapes include, but are not limited to, circles, ellipses, squares, triangles, polyhedrons, and ovals. Positions are generally uniform in size and the density of the positions on the substrates is at least 5/cm², 10/cm², 20/cm², 30/cm², 40/cm², 50/cm², 60/cm² 70/cm² 80/cm² 90/cm² 100/cm² 200/cm², 300/cm², 400/cm², 500/cm², 600/cm², 700/cm², 1000/cm², 5000/cm² or 10,000/cm². Preferably, the density of the positions on the substrates is at least 400-1000/cm².

In one embodiment of the invention, positions are ordered in the form of rows and columns. The total number of positions will vary depending on the number of different target nucleic acid molecules being monitored or identified. The number of positions on the array can range from 40 to 1000, 2,000, 2,500, 3,000, 3,500, 4000, 4,500, 5,000, 10,000, 50,000, 100,000, or even greater than about 250,000 different positions. In one embodiment a position comprises from 0.01 ng to .2 ng of nucleic acid, and preferably, 0.05 ng, in either single-stranded, double-stranded form, or partially double-stranded form (e.g., forming hairpins, or alternatively hybridized to other nucleic acids, primers, and the like).

In a further embodiment of the invention, the array comprises at least one control position. Control positions include, but are not limited to, positions comprising only buffer, a nucleic acid member which comprises a known sequence from the same organism as other nucleic acid members on the array, or from another organism. For example, in one embodiment, an array comprising human nucleic acid sequence members includes a control which is a known human gene (e.g., β-actin), while in another embodiment, an array comprising human nucleic acid sequences comprises at least one known non-human sequence (e.g., plant DNA, such as Arabidopsis thaliana DNA) belonging to a genetic pathway not found in humans. In still a further embodiment of the invention, multiple control positions are provided, including: a buffer only position, a human known sequence position, and a non-human sequence position. In one embodiment of the invention, substrate positions are provided which are stably associated with sequences which will hybridize to target molecules in any sample, and which are placed at asymmetric locations on the array to orient the relative positions of nucleic acid members on the array. In another embodiment of the invention, the orienting positions comprise total genomic DNA or poly dT oligonucleotides.

In one embodiment of the invention, each nucleic acid sequence has a unique and known position on the substrate with which it is stably associated. In another embodiment, nucleic acid members comprise both unknown and unknown sequences (with respect to publicly available databases) and each nucleic acid member is identified as a known or unknown sequence prior to being stably associated with the substrate. In a further embodiment of the invention, information relating to whether a nucleic acid member is known or unknown is stored within the memory of a computer or a computer program product along with information relating to the position of the nucleic acid member on the substrate of the array. In still a further embodiment of the invention, information relating to whether the sequence comprises a polyA sequence is also stored within the memory of a computer or computer program product.

Methods of Generating cDNA Arrays

In one embodiment, the invention provides a method of producing a cDNA array comprising noncoding sequences present at the 3′-ends of RNA transcripts. The method comprises selecting a cDNA sequence at random from a population of cDNA sequences (e.g., from a cDNA clone library, or a population of reverse transcription products, or RNA amplification products). In one embodiment, the population of cDNA sequences comprises a high representation of full-length clones. The sequence of at least a portion of the 3′-end of the cDNA is determined to identify a complementary sequence suitable for use as an amplification primer (e.g., a 3′-end PCR primer).

Amplification is performed by contacting a cDNA with the appropriate 3′-end primer, a polymerase, nucleotides, and an amplification buffer. The 3′-end primer is extended by the polymerase to generate a nucleic acid member which comprises the noncoding sequence present at the 3′-end of an RNA transcript corresponding to the cDNA. In one embodiment of the invention, the cDNA comprises at least one constant sequence (e.g., vector sequences or an adapter sequence) contiguous with a sequence at the 5′-end of the CDNA molecule and present in each cDNA in the population. A primer corresponding to the constant sequence end of the molecule is included in the amplification reaction to generate an amplified sequence which comprises the non-coding sequence present at the 3′-end of an RNA transcript corresponding to the cDNA and at least a portion of the constant sequence. Amplification methods are known in the art and include, but are not limited to, PCR using single or multiple primers, self sustained sequence replication (Guatelli et al., Proc. Natl. Acad. Sci. USA 87: 1874-1878, 1990), transcriptional amplification (Kwoh, et al., Proc. Natl. Acad. Sci. USA 86: 1173-1177, 1988), Q-Beta Replicase (Lizardi et al., Bio/Technology 6: 1197, 1988), ligase chain reaction (LCR) (see Wu and Wallace, Genomics 4: 560, 1989, Landegren et al., Science 241: 1077 (1988)), nucleic acid based sequence amplification (NASBA), and the like.

In one embodiment of the invention, a cDNA template is treated to remove repeat sequences (for example Alu sequences). According to this embodiment of the invention, the Alu sequence is identified according to methods well known in the art, and the template is amplified such that the Alu sequence is not included in the amplification product. For example, if the Alu sequence is 400 nucleotides upstream of the poly A tail, a primer is designed to hybridize with a sequence located, for example, approximately 390 nucleotides upstream of the poly A tail, so that the Alu sequence is not included in the amplified product. If the Alu sequence is located immediately adjacent to the poly A tail, two gene-specific primers, both located upstream of the Alu sequence, are designed and used for amplification.

Alternatively, if the Alu sequence is present in the amplified product, hybridization to the Alu sequences is blocked by including a highly repetitive blocker DNA in the hybridization buffer.

In another embodiment, a CDNA array is produced in which nucleic acid members comprise the non-coding sequence present at the 5′-end of an RNA transcript. The method comprises selecting a cDNA sequence at random from a population of CDNA sequences. The sequence of at least a portion of the 5′-end of the CDNA is determined to identify a complementary sequence suitable for use as an amplification primer (e.g., a 5′-end PCR primer). Amplification is performed by contacting the cDNA with the 5′-end primer, a polymerase, nucleotides, and an amplification buffer. The 5′-end primer is extended by the polymerase to generate a nucleic acid member which comprises the non-coding sequence present at the 5′-end of an RNA transcript corresponding to the cDNA. In another embodiment of the invention, the CDNA further comprises at least one constant sequence (e.g., vector sequences or an adapter sequence) contiguous with a sequence at the 3′-end of the CDNA molecule and present in all of the cDNAs in the population, and a primer corresponding to the constant sequence end of the molecule is included in the amplification reaction to generate an amplified sequence which comprises the non-coding sequence present at the 3′-end of an RNA transcript corresponding to the cDNA and at least a portion of the constant sequence.

In a preferred embodiment of the invention, the cDNA sequence contains substantially non-coding sequences from either the 5′-end or the 3′-end of a transcript (e.g., produces less than 50% of a full length polypeptide encoded by a gene corresponding to the transcript and excludes repeat elements (e.g., Alu elements). In one embodiment of the invention, the cDNA sequence comprises less than 45%, less than 40%, less than 30%, less than 20%, less than 10%, or less than 5% of the full length protein encoded by the RNA molecule.

By substantially excluding coding sequences and repeat sequences, the hybridization specificity of the array is enhanced, minimizing the chance that a nucleic acid member in a given position will cross-hybridize to target nucleic acid molecules which are less than fully complementary with the nucleic acid member (e.g., such as target nucleic acid molecules belonging to the same family of sequences as the one to which the nucleic acid member belongs).

In one embodiment of the invention, the sequence information obtained from at least a portion of the 3′-end of the CDNA or the at least a portion of the 5′-end of the DNA sequence is compared to sequence information in a public database. In one embodiment, 300-600 bases from the 3′-end or the 5′-end (as appropriate) of a cDNA is sequenced in a single pass. Sequence information obtained for each cDNA is compared to sequence information in public databases (e.g., available to anyone using a device connectable through the network without payment of a subscription fee) using a search tool to identify cDNAs having substantial sequence identity to one or more sequences in the database.

The term “substantial sequence identity” in the context of two or more nucleic acid sequences refers to one or more sequences or subsequences that have at least 95% percent identity over a comparison window consisting of a specified number of nucleotides after having been compared and aligned for maximum correspondence using a sequence comparison algorithm, or, alternatively by manual alignment and visual inspection. In one embodiment, a sequence having substantial sequence identity is a sequence which has at least 95% nucleotide sequence identity to a sequence in the database (a reference sequence) when aligned for maximum correspondence over a comparison window of 100 contiguous nucleotides, and preferably, 50-600 nucleotides. In a further embodiment of the invention, the sequence has at least 97% identity to the reference sequence when aligned for maximum correspondence over 200 nucleotides. Preferably, the sequence has 100% identity to the reference sequence when aligned for maximum correspondence over 200 nucleotides.

Methods of alignment of sequences for comparison are well-known in the art. Optimal alignment of sequences can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., supra). Multiple sequence alignments can be performed from a group of related systems using the PILEUP algorithm, which can be obtained from the GCG sequence analysis software package, e.g., version 7.0 (Devereaux et al., Nuc. Acids Res. 12:387-395 (1984).

Search tools such as the Basic Local Alignment Search Tool (“BLAST”) can also be used to identify cDNAs having substantial sequence identity to one or more sequences in a public database. Software for performing BLAST analyses is publicly available through the National Center for Biotechnology Information (http://www.ncbi.nlm.nih.gov/). The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered substantially identical to a reference sequence if the smallest sum probability in a comparison of the cDNA to the reference nucleic acid is less than about 0.001.

If a cDNA is identified as substantially identical to a known sequence in a public database, it is assigned an identifier which is the name and the accession number of the sequence with which it is substantially identical. In the case of a cDNA which represents the transcript of a human gene, it is also assigned a UniGene number (http://www.ncbi.nlm.nih.gov/UniGene and August 1996 NCBI News) if one is available. cDNAs which comprise subsequences which have substantial identity to one or more EST sequences in public databases are also assigned an EST number. cDNAs not having substantial identity to a sequence in a public database, are assigned an identifier designating the sequence as unknown and which is correlated in an array database with all available data relating to the sequence (e.g., sequence information, expression pattern, putative open reading frames, and motifs). In one embodiment of the invention, the user is provided with access to the array database when the user obtains the array.

Search tools also include the Basic Local Alignment Search Tool 2 (“BLAST 2”) used to align two given sequences and thereby identify regions having substantial sequence identity. Software for performing BLAST 2 analyses is publicly available through the National Center for Biotechnology Information (http://www.ncbi.nlm.mnh.gov/). The BLAST algorithm performs a statistical analysis of the similarity between the two sequences provided (Tatiana A. Tatusova, Thomas L. Madden (1999), “Blast 2 sequences—a new tool for comparing protein and nucleotide sequences”, FEMS Microbiol Lett. 174:247-250). Measures of similarity provided by the BLAST algorithm are the ‘bit’ score and Expect value. The ‘bit’ score, is defined as:

S′ (bits)=[lambda * S (raw)−1n K]/1n 2

where lambda and K are Karlin-Altschul parameters. The expression of the score in terms of bits makes it independent of the scoring system used. The Expect value estimates the statistical significance of the match, specifying the number of matches, with a given score, that are expected in a search of a database of this size absolutely by chance. An Expect value of two, with a given score, indicates that two matches with this score, are expected purely by chance. The Expect value changes with the size of the database (in a larger database more chance matches with a given score are expected), and is the most intuitive way to rank results or compare the results of one query run against two different databases. Also provided is an alignment of the two given sequences in the region of identity. The alignment indicates the number of identical nucleotides and the number of nucleotides in the region of identity. From these values, the % nucleotide identity in the region of identity is calculated.

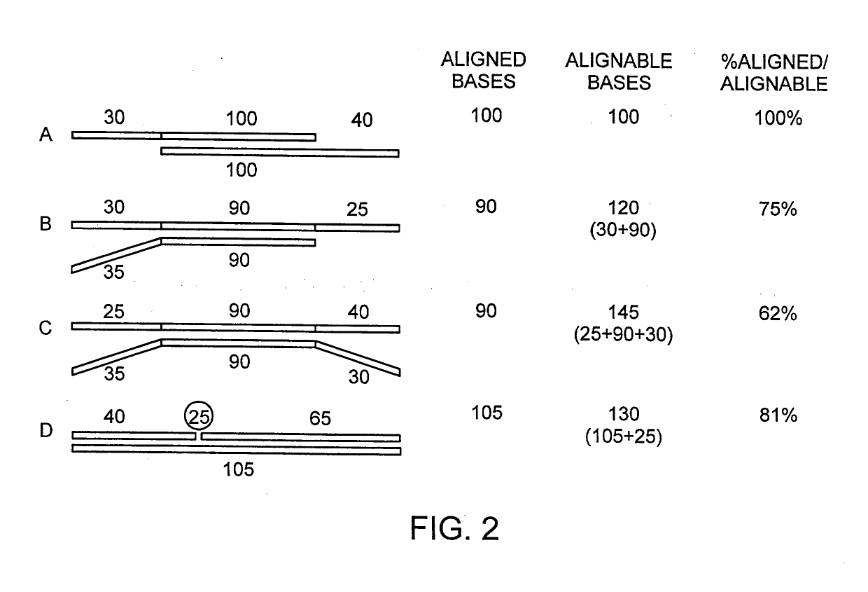

In one embodiment of the invention, a clustering algorithm is used to classify sequences as known or unknown and/or for sequence annotation (for example, described in Strategies, 2000, Volume 13, No.: 3, p. 93, Schuler et al., 1996, Science, 274:540-546; Miller et al., 1999, Genome Res., 9:1143-55; Burke et al., 1999, Genome Res., 9:1125-42; Burke et al., 1998, Genome Res., 8:276-90; Quackenbush et al., 2000, Nucleic Acids Res., 28:141-5; Garg et al., 1999, Genome Res., 9:1087-92; Wolfsberg et al., 1997, Nucleic Acids Res., 25:1626-32; Liang et al., 2000, Nucleic Acids Res., 28:3657-65; Liang et al., 2000, Nat. Genet., 25:239-40; Eckman et al., 1998, Bioinformatics, 14:2-13; Miller et al., Genome Res., 1997: 1027-32; Jiang et al., 1998, Genome Res., 8:268-75, herein incorporated by reference in their entirety). In another embodiment of the invention, sequences in a cDNA being characterized are compared with sequences in a database to identify shared sequence elements. The CDNA is then compared with a sequence having a shared sequence element(s) identifying regions of local alignment of sequences flanked by unaligned sequences (see FIG. 2). At each end of a local alignment, the shorter of the two unaligned sequences is used to calculate the number of alignable bases, which is the sum of the local alignment length plus the length of unaligned sequence flanking the local alignment (FIGS. 2). In a preferred embodiment of the invention, a CDNA is identified as substantially identical to a sequence in the database if the percentage of alignable sequences is greater than 90%.

In this analysis, alignments of commonly repeated or low complexity sequences are discarded (e.g., Alu sequences). Sequences from different gene family members will not be clustered since the untranslated regions of these sequences do not tend to align. Sequencing artifacts are ignored because they generally do not cause the identity of a sequence to drop below 95% of its true identity. In one embodiment, only cDNAs comprising polyA tails are used to perform the analysis and only sequences immediately adjacent to the polyA tail are compared. This eliminates the possibility of counting the expression product of a single gene multiple times, and minimizes the possibility that RNA splice variants of a single transcript will be counted as multiple different expressed genes. Alternatively, the clustering algorithm may be modified to ignore splice variants by eliminating internally unpaired sequence from the computation of the alignable length (see FIG. 2D). This clustering method provides a more accurate estimate of the number of different genes represented by the population of cDNAs amplified.

In one embodiment, at least 2% of the population of cDNA molecules used to generate the CDNA array, does not contain significant sequence identity to a nucleic acid sequence in a public database. In other embodiments, at least 5%, 10%, 15% or 20% of the population of cDNA molecules used to generate the cDNA array, does not contain significant sequence identity to a nucleic acid sequence in a public database.

The process of cDNA selection, generation of nucleic acid members, and identifying nucleic acid members as known or unknown sequences, is repeated multiple times, either sequentially, or simultaneously, to generate a set of nucleic acid members, each nucleic acid member comprising a noncoding sequence present at either the 3′-end or 5′-end of an RNA transcript.

After having classified at least two nucleic acid member sequences as known or unknown, nucleic acid members are stably associated with a substrate at unique positions on the substrate, generating an array of cDNA sequences. In a preferred embodiment of the invention, nucleic acid members are examined by at least one quality control step to determine that there is really only one type of sequence per nucleic acid member, and that the identity of at least a portion of the sequence, has been classified properly as a particular known or unknown sequence. Quality control steps can include, but are not limited to, digestion of a nucleic acid member with a restriction enzyme and gel electrophoresis to verify that the nucleic acid member has the proper restriction enzyme digest pattern, and sequencing of all or a portion of the nucleic acid sequence (e.g., using a known sequence primer). In one embodiment, approximately, 300-600 nucleotides at either the 3′-end (if the nucleic acid member comprises 3′-end noncoding sequences) or at the 5′-end (if the nucleic acid member comprises 5-end noncoding sequences) of the nucleic acid member is sequenced to verify that the nucleic acid member comprises a single type of nucleic acid sequence and to confirm the identity of the nucleic acid sequence as a particular known or unknown sequence.

In one embodiment of the invention, the nucleic acid members on the substrate comprise human nucleic acid sequences and preferably at least 2% of the nucleic acid members on the substrate do not contain substantial nucleotide sequence identity to a nucleic acid sequence in a public database. In other embodiments, at least 5%, 10%, 15% or 20% of the nucleic acid members on the substrate do not contain substantial nucleotide sequence identity to a nucleic acid sequence in a public database. In another embodiment of the invention, the cDNA sequences comprise sequences from two or more tissues (e.g., human tissues), and preferably, at least 2% of the population of cDNA sequences do not contain significant nucleotide sequence identity to a nucleic acid sequence in a public database. In other embodiments of the invention, the cDNA sequences comprise sequences from two or more tissues (e.g., human tissues), and at least 5%, 10%, 15% or 20% of the population cDNA sequences do not contain significant nucleotide sequence identity to a nucleic acid sequence in a public database.

Method of Using CDNA Arrays for Gene Expression Monitoring

The invention further provides a method of analyzing the expression of one or more genes by hybridizing target nucleic acids to an array comprising either 3′-end noncoding sequences or 5′-end noncoding sequences. In one embodiment of the invention, samples are isolated or commercially obtained from a biological system, i.e., any of: a cell, a group of cells, a tissue, a group of tissues, an organ, or an organism (e.g., a unicellular or microscopic multicellular organism). Labels are attached to nucleic acids corresponding to RNA transcripts within the sample (“target nucleic acids”) and hybrids between these nucleic acids and the nucleic acid members on the array are detected by detecting the labels.

The incorporation of labels into target nucleic acids is well known in the art. In one embodiment of the invention, labels are added to transcripts in an in vitro transcription reaction, e.g., such as described by Schena, et al., Science 270: 467 (1995), the entirety of which is incorporated herein by reference. In another embodiment, 100 ng -20 μg of polyadenylated RNA (e.g., mRNA) is prepared from total RNA using a support to which oligo-dT is bound (e.g., Oligotex-dT resin (Qiagen) or oligo-dT magnetic beads (Dynal)). RNA transcripts are amplified, such as by reverse transcription (for example, using a Stratascript® RT-PCR kit), in the presence of labeled nucleotides. In a further embodiment, RNA ligase is used to incorporate labels directly into polyadenylated RNA (see, e.g., Richardson et al., “Biotin and Fluorescent Labeling of RNA Using T4 RNA Ligase,” Nuc. Acids Res., 11: 6167-6184,1983; U.S. Pat. No. 6,040,138, and U.S. Pat. No. 6,027,886, the entireties of which are incorporated herein by reference). In still another embodiment of the invention, total RNA is labeled.

Detectable labels suitable for use in the present invention include any composition detectable by spectroscopic, photochemical, biochemical, electrical, optical, or chemical means. Useful labels suitable for practicing the present invention, include, but are not limited to, biotin, streptavidin, fluorescent dyes (e.g., fluorescein, lissamine, Texas Redo, rhodamine, green fluorescent protein, BODIPY® dyes, and the like), radiolabels (e.g., ³H, ¹²⁵I, ²⁵S, ¹⁴C, ³²P, and the like), enzymes (e.g., horseradish peroxidase, alkaline phosphatase, and other enzymes commonly used in ELISA procedures), and colorimetric labels, such as colloidal gold or plastic (e.g., polystyrene, polypropylene, latex, and the like).

In one embodiment of the invention, the labeled target nucleic acids represent substantially all (at least 50%) of the transcripts within a biological system (cell, group of cells, tissue, group of tissues, organ, or organism), while in another embodiment of the invention, the labeled target nucleic acids represent a specific transcript or set of transcripts whose expression is being monitored. In this embodiment of the invention, label is incorporated into a specific target nucleic acid(s) by amplifying these target nucleic acid(s) using primers which hybridize specifically to the transcripts being monitored and not to other transcripts within the sample. Methods of amplifying RNA molecules using primer molecules are well known in the art, and, in addition to RT-PCR methods, include Self-sustained sequence replication amplification (3SR) (Fahy, et al. PCR Methods and Applications 1:25-33 (1991)), and a method that utilizes an oligo dT primer containing a phage T7 promoter, and provides for transcription of a cDNA molecule using a T7 RNA polymerase (described in U.S. Pat. No. 5,545,522, the entirety of which is incorporated by reference herein). RNA amplification methods can be performed alone, or in combination with other amplification methods, such as self sustained sequence replication (Guatelli et al., Proc. Natl. Acad. Sci. USA 87: 1874-1878, 1990), transcriptional amplification (Kwoh, et al., Proc. Natl. Acad. Sci. USA 86: 1173-1177, 1989), Q-Beta Replicase (Lizardi et al. Bio/Technology 6: 1197, 1988), ligase chain reaction (LCR) (see Wu and Wallace, Genomics 4:560,1989), Landegren et al., Science 241: 1077, 1988) and nucleic acid based sequence amplification (NASBA).

A sample comprising labeled target nucleic acids is then contacted with the array under conditions sufficient to allow specific hybridization to occur (e.g., each target labeled transcript molecule hybridizes to its complement and does not hybridize to noncomplementary sequences either in the sample or in the array itself). Suitable hybridization conditions are known in the art and are reviewed in Short Protocols in Molecular Biology, 4^{th} Edition, 1999, ed. Ausubel, et al., the entirety of which is incorporated herein by reference. In one embodiment of the invention, hybridization is performed for 12-24 hours at 42-65° C. in hybridization buffer (e.g., 2× SSC). One to a plurality of washes is then performed to remove any unbound molecules or nonspecifically bound molecules from the substrate. In a preferred embodiment of the invention, the array is treated prior to hybridization to minimize nonspecific binding of target molecules. In one embodiment, the array is treated with a solution of 1% “Blotto” or 50 mM tripolyphosphate, or other pre-hybridization solution, routinely used in the art, for at least one hour at 37° C.-50° C. In another embodiment of the invention, blocking nucleic acids are added to the prehybridization solution, e.g., an excess of Alu DNA or polyA oligonucleotides, Cot1 DNA (Human Cot-1 DNA, Life Technologies; Mouse Cot-1 DNA). In still another embodiment of the invention, the array is washed and stripped of bound target molecules (e.g., by boiling in water or 0.5% SDS) to enable reuse of the array.

Detection of hybridization is performed using methods which are appropriate for detecting the label used. In one embodiment, when a colorimetric label is used, hybridization is detected by visualizing the label. In another embodiment, when a radioactive label is used, radiation is detected (e.g., such as by phospho-imaging or autoradiography). In a further embodiment, target nucleic acid molecules are labeled with fluorescent labels and the localization of the label on the array is accomplished by phospho-imaging or by fluorescent microscopy. In one embodiment, the hybridized array is excited with a light source (e.g., a laser) at the excitation wavelength of the particular fluorescent label and the resulting fluorescence at the emission wavelength is detected.

In a further embodiment of the invention, an optical system is used to analyze hybridization signals on the array. The optical system comprises a monochromatic or polychromatic light source, a focusing system for directing excitation light from the light source to the array, and a detector for detecting fluorescent emissions from the array. In another embodiment of the invention, light is directed to a particular position, or positions, on the array through the use of a x-y-z translation table which can be controlled by a processor which also communicates with the detector. Light from the light source can also be focused to a specific size (e.g., number of positions) by controlling the dimension and placement of objective lens with respect to the light source and the array. The effects of the dimensions and placement of objective lens are well known in the art and are described in U.S. Pat. Nos. 5,760,951, 5,923,466, 5,923,466, 5,587,832, and 5,162,941, for example, the entireties of which are incorporated herein by reference.

In additional embodiments, the optical system comprises an auto-focusing mechanism to maintain the array in the focal plane of the excitation light from the light source throughout the excitation process. Temperature controllers can also be provided, to provide temperatures which maintain the stability of the hybrids formed on the array. In a further embodiment of the invention, the optical system comprises a confocal microscope which can perform multiple scanning operations within a single plane (see, e.g., U.S. Pat. No. 5,874,219, the entirety of which is incorporated by reference herein).

In other embodiments, an optical system is provided which is equipped with a phototransducer (e.g., a photomultiplier, a solid state array, charge-coupled devices (CCD) or charge-injection devices (CID), image-intensifier tubes, image orthicon tube, vidicon camera type, image dissector tube, or other imaging devices) attached to an automated data acquisition system to automatically record any fluorescent signal produced. These types of automated systems are known in the art (see, e.g., U.S. Pat. Nos. 5,143,854, 4,605,485, 5,692,507, and 3,743,768, the entireties are incorporated herein by reference).

In one embodiment of the invention, the detector comprises a CCD imaging system which can be used in combination with filter elements and/or optical fibers to limit light reaching the detector to the fluorescent light which is emitted by the array. In another embodiment, a CCD device is provided which is in proximity to the substrate (e.g., within 1-2 cm of the substrate); while in another embodiment, the CCD device is an integral component of the substrate forming the array. In this embodiment, the CCD detector comprises an array of discrete devices, each of which is a “pixel” for storing charge which is representative of emitted light from the array. The number of pixels provided in the CCD array is optimized to sufficiently detect an image produced by the collection optics of the optical system being used with the cDNA array and will vary depending on the number of positions in the cDNA array (see, e.g., U.S. Pat. Nos. 6,045,996, 5,874,219, and 6,025,601, the entireties of which are incorporated herein by reference).

CCD arrays suitable for imaging a variety of different sized arrays are available commercially and include those from DALSA, Inc. (Easton Conn.), David Samoff Research Center (Princeton, N.J.) or Princeton Instruments (Trenton, N.J.)). Other detector arrays which are encompassed within the scope of the invention, include, but are not limited to, an intensified CCD array (such as that available from Princeton Instruments, Hamamatsu Corp., Bridgewater, N.J. or Photometrics Ltd.,Tucson, Ariz.), a focal plane array (such as that available from Scientific Imaging Technologies, Inc., Beaverton, Oreg.), Eastman Kodak Co., Inc. (Rochester, N.Y., or David Sarnoff Research Center), a photodiode array (such as that available from Reticon Corp., Sunnyvale, Calif., Sensors Unlimited, Inc., Princeton, N.J., or Hamamatsu) or a photodetector array (such as that available from FLIR Systems Inc., Portland, Oreg., Loral Corp., New York, N.Y., or Hughes Electronic Corp., Los Angeles, Calif.).

In another embodiment of the invention, the optical system comprises excitation optics which focuses excitation light to a line on the cDNA array and scans a plurality of lines by using a translation stage that moves at a constant velocity (see, e.g., U.S. Pat. No. 5,557,113). Collection optics receive light emitted by the scanned cDNA array and transmits the received light onto a linear array of light detectors. In this way, signal data relating to a plurality of one dimensional images is obtained. By adding rotating mirrors to the system, 2- and 3-dimensional images can also be obtained.

In an alternative embodiment of the invention, hybridization is detected without the use of labels, for example by placing capacitors contiguous to each cDNA position or by forming a transmission line between two electrodes at each cDNA position, to measure changes in AC conductance or radiofrequency loss, respectively, upon hybridization of a target molecule to the cDNA at that position (see, e.g., U.S. Pat. No. 5,843,767 and WO 93/22678, the entireties of which are incorporated by reference herein).

It should be obvious to those of skill in the art that a variety of detection systems as discussed above can be used to analyze the hybridization of target nucleic acid molecules to the array. The choice of system is dictated, in part, by the sensitivity and speed desired by the user. One of skill in the art will appreciate that methods for evaluating hybridization results will vary with the nature of the labels employed. When using fluorophore labels, the amount of signal corresponding to a hybridization event can be maximized by optimizing both the fluorophore and the amount of excitation energy from the light source being used. For example, for fluorescein, a good signal-to-noise ratio can be obtained using a CCD detector in combination with a 488 nm Argon laser which provides light at 3 mW/cm² in 30 seconds. By increasing laser power and using labels which are less sensitive to photodestruction and whose emission more closely matches the sensitivity maximum of the CCD detector (e.g., dyes like CY3 or CY5), the sensitivity and speed of detection can be enhanced (see, e.g., U.S. Pat. No. 6,025,601).

In one embodiment, the amount of label at a selected position is determined and compared with the amount of label detected at each position on the array (e.g., at each spot), including control positions (i.e., where no nucleic acid members are present or where known sequences are present). The amount of label after correcting to subtract background signal, is proportional to the expression level of a target nucleic acid which corresponds to the nucleic acid member stably associated with that position. In one embodiment of the invention, the array is addressed (e.g., the identity of a nucleic acid member at a given position is known). In this embodiment, a processor transforms data relating to fluorescent emissions into substrate position data after removing outliers (data relating to positions which emit fluorescence, but whose signals fall below a pre-selected acceptable intensity, based upon routine statistical determinations of expected distributions of intensity).

The amount of background signal and the specificity of hybridization can also be determined by monitoring signal obtained at control position(s) on the array. In one embodiment, a CDNA array comprising human nucleic acid members includes multiple control positions. In this embodiment, at least one control position comprises only buffer, at least one control position comprises a “housekeeping gene CDNA,” e.g., a known human CDNA sequence corresponding to a gene whose expression does not significantly differ between several tissues examined (e.g., β-actin sequence). In another embodiment, at least one control position comprises non-human sequences for which there should be no target molecules in the sample (e.g., plant sequences, such as Arabidopsis thaliana sequences). A positive signal corresponding to the housekeeping gene position indicates that hybridization conditions were appropriate to detect at least this sequence in a population of target nucleic acid molecules.

The position comprising buffer and the position comprising non-human sequences should not provide a detectable signal or should provide an acceptable background signal (e.g., one which is significantly different from the signal produced by the housekeeping gene sequence, to within 95% confidence levels, as determined by standard statistical measures). By contacting the array with samples which do not contain the housekeeping sequence and which are spiked with different known amounts of the housekeeping sequence, a correlation can be obtained between the amount of signal observed and the amount of target nucleic acid in a sample.

The stringency of hybridization conditions can be optimized by determining the kinetics of hybridization, i.e., by measuring the mount of binding at each of a number of different time points. This allows the user to determine the dependency of the hybridization rate for different cDNAs on temperature, sample agitation, washing conditions (e.g. pH, solvent characteristics, temperature), and the like. The speed with which CCD imaging systems operate make these systems ideal for determining hybridization kinetics (see, e.g., as described in Fodor et al., U.S. Pat. No. 5,324,633, incorporated herein by reference).

In one embodiment, data obtained from a hybridization reaction (e.g., optical data from labeled nucleic acid members on the array) are displayed as an image on the display of a device connectable to the network (e.g., a computer or wireless device), for example, using color to demonstrate regions of high intensity signal vs. regions of low intensity signal. In another embodiment of the invention, data relating to a signal includes information relating to the substrate position associated with the signal. In a further embodiment of the invention, data relating to the identifier assigned to a cDNA stably associated with a particular substrate position is displayed.

In still another embodiment, the user is provided with a display which is part of an interface on a device connectable to the network, and the user is provided with a plurality of selectable options (e.g., buttons on the interface or links) for accessing information relating to the displayed signal.

In one embodiment of the invention, the information includes the substrate position on the array of the nucleic acid member which is labeled and is being detected. In another embodiment, the information includes the name of the identifier associated with the nucleic acid member. In still a further embodiment, the information includes information relating to the cDNA associated with the identifier (e.g., known or unknown, tissues in which the cDNA is expressed, any association with disease, restriction digest pattern, putative open reading frames, and the like). In still another embodiment of the invention, the resulting data is displayed as an image with color in each region varying with the light emission or binding affinity between targets and probes therein. In a further embodiment of the invention, an image of a restriction enzyme digest of the cDNA and/or a map or schematic diagram indicating the position restriction sites relative to nucleotide position on the sequence are displayed

While a preferred embodiment of the invention contemplates the use of a data processor to determine substrate positions relative to an observed or measured signal, in another embodiment of the invention, information related to the identification of cDNAs at particular substrate positions (e.g., such as the cDNA identifiers) is provided to the user in the form of written information (e.g., typed, handwritten, faxed, or printed from a computer) and can further include information relating to the sequence of the cDNA at a particular substrate position. In still a further embodiment of the invention, a URL is provided to the user which allows the user to access a database containing information relating to the cDNAs on the array.

By determining whether any expressed target nucleic acid sequence (e.g., mRNA) within the sample hybridizes to the array, data relating to the expression of the target nucleic acid sequence is obtained. In one embodiment of the invention, the data comprises the amount of target nucleic acid sequence expressed in a sample. In another embodiment of the invention, the data comprises the identity of the nucleic acid member to which the target nucleic acid sequence hybridizes (e.g., a known or unknown sequence). In still another embodiment of the invention, a nucleic acid member comprising an unknown sequence which has hybridized to a target nucleic acid sequence is sequenced. In a further embodiment of the invention, the sequence of the unknown sequence is entered into the memory of a computer or a computer program product and the sequence is identified as. a known sequence and information about its expression pattern is entered into the memory of the computer or computer program product.

In still a further embodiment of an invention, an expression profile is generated comprising data related to the expression of a gene or group of genes in a biological system (e.g., a cell, group of cells, tissue, group of tissues, organ, or organism) in healthy and pathological states (where the biological system is subject to genetic alterations and/or environmental disturbances) using the arrays of the invention. In a further embodiment of the invention, normalized data relating to the expression profile of a plurality of the same biological systems are stored in the memory of a computer or a computer program product.

In another embodiment, the effects of a particular drug or set of drugs on gene expression is monitored. In this embodiment, a drug or set of drugs is administered to a biological system (e.g., cells, group of cells, tissue, group of tissues, organ, or organism) and labeled target nucleic acids from the biological system are prepared as described above, along with labeled target nucleic acids from an untreated biological system. By comparing the expression profile of a target nucleic acid (or plurality of target nucleic acids) in the treated and untreated systems, the efficacy of a drug may be monitored.

In a further embodiment of the invention, the biological system comprises a pathology and the expression profile of the treated biological system is compared to the expression profile of a healthy biological system. In a further embodiment of the invention, the expression profile of the treated biological system is also compared to the expression profile of the untreated biological system having the pathology. In another embodiment of the invention, the expression profile of the treated biological system is compared to normalized data relating to the expression profile of healthy biological systems and systems comprising a pathology, and the dosage of the drug (or sets of drugs) is altered based on this comparison (e.g., no more drug is provided if the treated profile substantially resembles the untreated profile, such that there is no significant difference between the profiles to within 95% confidence levels).

Gene Discovery Using cDNA Arrays

As described above, the arrays of the invention represent both known and unknown genes because the cDNAs used to generate the nucleic acid members are selected at random from a population of cDNA comprising both known and unknown sequences. In one embodiment of the invention, the population comprises at least 15% unknown sequences, and preferably 20-50% unknown sequences. The analysis of gene expression using the cDNA arrays of the invention therefore provides a method of gene discovery as the expression of previously unknown genes can be detected and quantitated.

In one embodiment, the biological relevance of a previously unknown or uncharacterized gene is determined by determining the expression profile of this gene in a biological system. In still another embodiment, the expression profile of a previously unknown or uncharacterized gene is compared to the expression profile of other genes. In still a further embodiment, compared profiles are used to identify interactions between genes. In one embodiment of the invention, the user of the array can search a database (e.g., provided through a server) which they can access using a device connectable to the network (e.g., a user computer or wireless device). In this embodiment, a search engine is also accessed which can search the database for sequences sharing common sequence motifs or similar expression patterns to the nucleic acid member. In another embodiment of the invention, the sequence of an unknown cDNA identified as being of interest is translated into all six reading frames, and the sequence is compared again to all sequences in publicly available databases to update the previous search that was done in generating the array and to identify any sequence similarities between the unknown cDNA and the sequences in the database.

### EXAMPLE I

Production of Human cDNA Microarrays Comprising the 3′-End Noncoding Sequences of RNA Transcripts

Microarrays of 3′ cDNA sequences have been constructed from libraries of human cDNAs contained in Stratagene's GeneConnection™ clone collection. This collection consists of clones from innovative libraries that contain a high number of clones (about 20%) that do not have significant nucleotide homology to clones in public databases. Moreover, these libraries represent clones from 29 different human tissues, including, adrenal gland, bone marrow, brain (whole amygdala, caudate nucleus, cerebellum, hippocampus, substantia nigra, subthalmic nuclei, thalamus), heart, kidney, liver, lung, lymph node, mammary gland, pituitary gland, placenta, prostate, skeletal muscle, small intestine, spinal cord, spleen, testis, thymus, thyroid, trachea, and uterus.

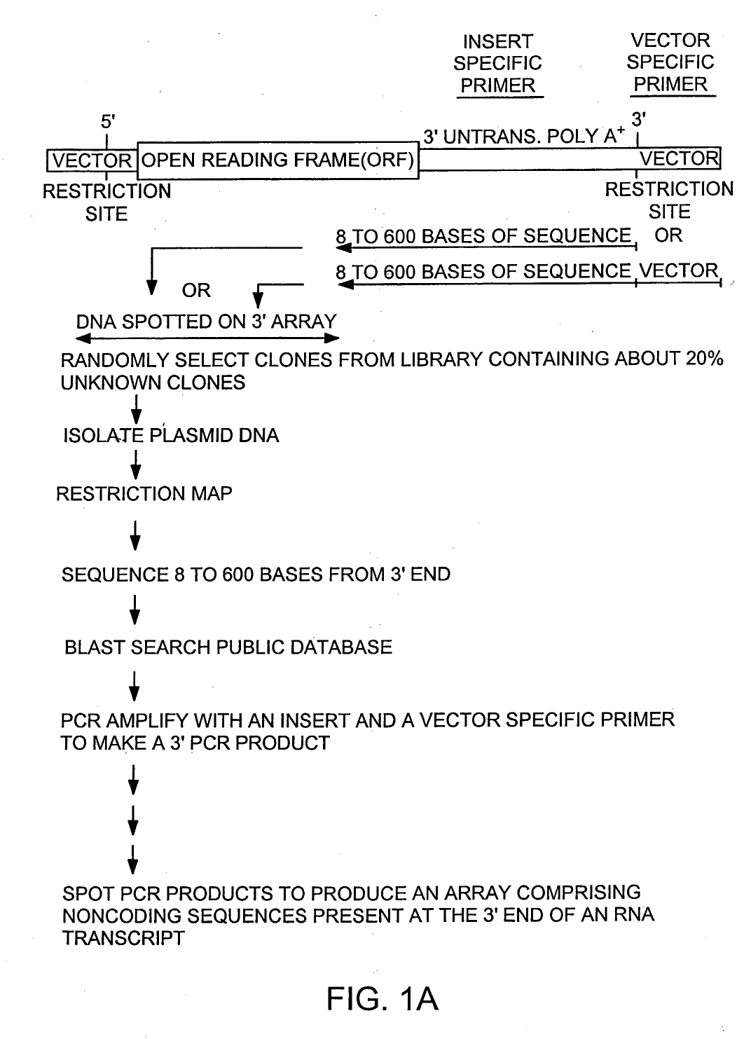

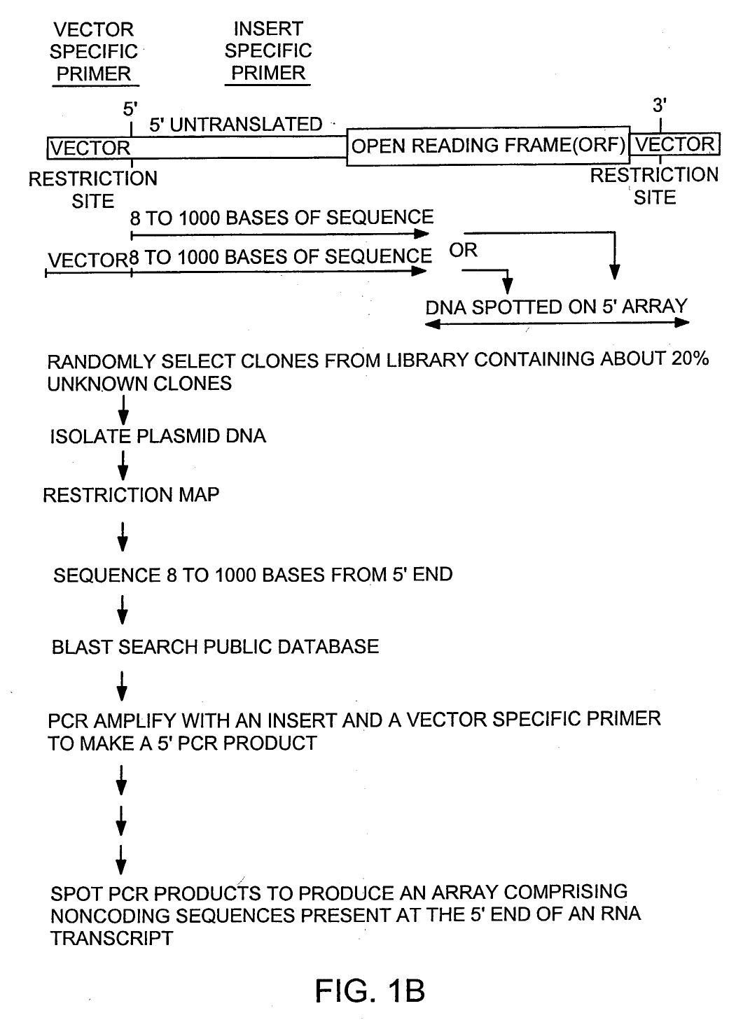

The human cDNA microarray is produced from clones selected at random from the clone collection, as diagrammed in FIG. 1A. Plasmid DNA of each clone is isolated by means known in the art. The purity of each plasmid is examined by restriction mapping, using restriction enzymes such as SacI, HindII, and SacI combined with HindIII or any other enzymes which generate an informative pattern (e.g., unique to a particular plasmid). The restricted DNA is analyzed by gel electrophoresis alongside uncut, supercoiled plasmid. The DNA in the gel is visualized by ethidium bromide staining, and an image of the gel is captured (e.g., by a photgraph). The purity of the plasmid is further determined by sequencing approximately 300-600 base pairs of the 3′ end of the cDNA insert with a vector-specific primer.

Based on the 3′ sequence information, an insert-specific primer (e.g., complementary to at least a portion of the 3′-end) is selected (either synthesized or obtained commercially) after identifying (either visually or using a computer program, such as BLAST) a 3′-end primer sequence (insert-specific primer) which will specifically amplify approximately 350 bases of the 3′ end of the cDNA, including the polyA tail. In one embodiment of the invention, PCR is performed using two primers, the 3′-end primer sequence and a vector specific primer complementary to a vector sequence on the strand of the vector which is opposite to the strand from which the 3′-end primer sequence is obtained. After PCR with the insert-specific and vector-specific primers, the presence of a single PCR product of the correct length is confirmed by gel electrophoresis. If the cDNA template contains minor amounts of contaminating DNA, such DNA will not amplify with the insert-specific primer. Moreover, if the cDNA templates have been inadvertently mixed-up in a prior step, a PCR product of the predicted length will not be amplified. Thus, PCR with an insert-specific primer both purifies and confirms the identity of the cDNA.

By substantially excluding coding sequences from the PCR product by selecting for PCR products which are less than 600 nucleotides, not including any vector or adaptor sequences at the 5-end of the PCR product, PCR products are selected which comprise substantially noncoding sequences. If the PCR products contain repeat sequences (for example Alu sequences), the repeat sequences are removed according to the methods described in the section entitled “Methods of Generating CDNA Arrays” (above). Hence, this design increases hybridization specificity when using the 3′-end cDNA array by minimizing the chances that a nucleic acid member in any given position will cross hybridize with RNA-derived probes from other gene family members or with sequences comprising repeat elements.

The increase in hybridization specificity when using this design was demonstrated by using the BLAST algorithm. BLAST 2 was used to align the nucleotide sequences of the coding regions of several cytochrome p450 family members to identify regions of significant identity. The 3′ UT regions were also analyzed using BLAST 2. The cytochrome p450 family members consist of a superfamily of more than 160 known members that play a major role in the metabolism of numerous physiological substrates.

Several cytochrome p450 family members were identified in the GeneConnection clone collection. They included CYP2A7, CYP4B1, CYP4F8, CYP11A, and CYP4A11. BLAST comparisons were made between the nucleotide sequences of each of these family members in the GeneConnection database and the blast nr database to identify the NCBI Reference Sequence for each family member (Table A). The nucleotides representing the coding and 3′ untranslated regions of the NCBI Reference sequences were identified from the information in NCBI related to each of the cytochrome p450 family members.

TABLE A
Cytochrome p450 family members

Nucleotide Sequence Position

| Name | NCBI Reference Sequence | Coding | 3′UTR |
|---|---|---|---|
| CYP2A7 | NM_000764 | 544-2028 | 2029-2282 |
| CYP4B1 | NM_000779 | 13-1548 | 1549-2084 |
| CYP4F8 | NM_007253 | 8-1570 | 1571-1587 |
| CYP11A | NM_000781 | 45-1610 | 1611-1821 |
| CYP4A11 | NM_000778 | 42-1601 | 1602-2470 |

The nucleotides representing the coding regions of each of the NCBI Reference sequences were then compared in a pairwise manner using BLAST 2 to identify regions of significant sequence identity. The 3′ UT regions were also compared. The results of these comparisons are given in Tables B and C. Results of the comparisons between the 3′ UT regions are in the upper part of the table above the cells containing the horizontal line (-------). Results of the comparisons between the coding regions are in the lower part of the table below the cells containing the horizontal line (-------). Pairwise comparisons that did not identify regions with significant identities are indicated in Tables B and C as none. When the pairwise comparison identified regions with significant identity, the % identity is given in Table B. When more than one region of identity is identified, the values for each of the regions is indicated. The number of bases of identity divided by the number of bases in the region of identity for each of the regions is given in parenthesis following the % identity. Table C gives the bit scores and Expected values for each pairwise comparison. The bit scores are first and the Expected values are second for each of the regions of identity. The bit scores and Expected values in Table C are separated by a comma.

TABLE
B. % identities of coding and 3′ UT regions
of cytochrome p450 family members

| | CYP2A7 | CYP4A11 | CYP4B1 | CYP4F8 | CYP11A | |
|---|---|---|---|---|---|---|
| CYP2A7 | — | none | none | none | none | 3′UT |
| CYP4A11 | none | — | none | none | none | |
| CYP4B1 | none | 87% (67/77) | — | none | none | |
| | | 80% (76/95) | | | | |
| | | 72% (81/111) | | | | |
| CYP4F8 | none | 85% (35/41) | 84% (61/72) | — | none | |
| | | 76% (54/77) | 81% (65/80) | | | |
| CYP11A | none | none | none | none | — | |

coding

TABLE C
Bit scores and Expected values of coding
and 3′ UT regions of cytochrome p450
family members

| | CYP2A7 | CYP4A11 | CYP4B1 | CYP4F8 | CYP11A | |
|---|---|---|---|---|---|---|
| CYP2A7 | — | none | none | none | none | 3′UT |
| CYP4A11 | none | — | none | none | none | |
| CYP4B1 | none | 91.1, 4e−16 | — | none | none | |
| | | 73.7, 7e−11 | | | | |
| CYP4F8 | none | 44.9, 0.032 | 75.7, 2e−11 | — | none | |
| | | 39.1, 1.8 | 68.0, 4e−09 | | | |
| CYP11A | none | none | none | none | | |

coding

The results in Tables B and C indicate that significant identities were identified between several of the coding regions of the cytochrome p450 family members, however, no significant identities were identified in the 3′ UT regions. The cytochrome p450 family members having significant identity in the coding regions were CYP4A11 and CYP4B1, CYP4A11 and CYP4F8, and CYP4B1 and CYPA4F8.

The 3′ cDNA PCR products (nucleic acid members) are stably associated with a substrate which is a standard 25 mm X 75 mm glass microscope slide either by an arrayer or manually as described above. The array substrate thus comprises a plurality of positions, each position comprising a different nucleic acid member. In one embodiment, each position is in the form of a spot. In another embodiment, the array comprises more than 4,000 human cDNA sequences spotted in a 44×96 grid, with each cDNA sequence spotted at a unique, predetermined location on the grid. The array is then used in methods known in the art or in the methods described above, to profile gene expression and discover new genes.

Profiling of gene expression is facilitated because information is available for each cDNA sequence spotted on the array. Following sequencing of the 3′ end of cDNAs from the clone collection (as described above), the sequences are compared to those in public databases using the BLAST algorithm described above. Clones having substantial identity to one or more characterized sequences in public databases are assigned a name, accession number, and UniGene number. Clones comprising a sequence or subsequence having significant identity to one or more Expressed Sequence Tag (EST) sequences in the public databases are also assigned an EST number. Clones not having significant nucleotide homology to those in the public databases are identified as “unknown” and are maintained in a database accessible to users of the micro arrays.

Several methods are available to identify and evaluate the clones in the clone collection. The collection can be searched for a specific clone by using a gene name, accession or UniGene number, nucleotide sequence, or location on a 3′ cDNA microarray. Additional information available includes gel images of restriction enzyme digestions of individual clones and gel images demonstrating the length and purity of PCR products used for microarray spotting.

### EXAMPLE 2

Production of Human cDNA Microarrays Comprising the 5′-End Noncoding Sequences of RNA Transcripts

Microarrays of 5′-end cDNA sequences are constructed using techniques routinely used in the art (e.g., 5′ RACE, random priming or oligo dT priming and size selection of RNAs, CapFinder PCR cDNA Library Construction) or using commercially available libraries (e.g., CLONTECH's 5′-STRETCH PLUS cDNA Libraries ). cDNAs containing 5′-end noncoding sequences can also be obtained by size selecting for longer clones (according to methods well known in the art), and sequencing the resulting clones. Alternatively, cDNAs containing 5′-end noncoding sequences, but lacking sequence that is not a “sequence at the 5′ end”, as defined hereinabove, are obtained by using two gene-specific primers for cDNA isolation.

In one embodiment, a human cDNA microarray is produced from clones selected at random from a clone collection enriched in 5′-non-coding sequences, as diagrammed in FIG. 1B. Plasmid DNA of each clone is isolated and characterized as described above in Example 1. The purity of the plasmid is further determined by sequencing approximately 300-600 base pairs of the 5′ end of the cDNA insert with a vector-specific primer.

Based on the 5′ sequence information, an insert-specific primer (e.g., complementary to at least a portion of the 5′-end) is selected (either synthesized or obtained commercially) after identifying (either visually or using a computer program, such as BLAST) a 5′-end primer sequence (insert-specific primer) which will specifically amplify approximately 350 bases of the 5′ end of the cDNA. In one embodiment of the invention, PCR is performed using two primers, the 5′-end primer sequence and a vector specific primer complementary to a vector sequence on the strand of the vector which is opposite to the strand from which the 5′-end primer sequence is obtained. After PCR with the insert-specific and vector-specific primers, the presence of a single PCR product of the correct length is confirmed by gel electrophoresis. If the cDNA template contains minor amounts of contaminating DNA, the DNA will not amplify with the insert-specific primer. Moreover, if the cDNA templates have been inadvertently mixed-up in a prior step, a PCR product of the predicted length will not be amplified. Thus, PCR with an insert-specific primer both purifies and confirms the identity of the cDNA.

By substantially excluding coding sequences from the PCR product by selecting for PCR products which are less than 1000 bp, not including vector sequences at the 5′-end of the PCR product, PCR products are selected which comprise substantially noncoding sequences, minimizing the chances that the DNA in any given spot will cross hybridize with RNA-derived probes from other gene family members or with repeat elements. If the PCR products contain repeat sequences (for example Alu sequences), the repeat sequences are removed according to the methods described in the section entitled “Methods of Generating cDNA Arrays” (above).

The 5′-end cDNA PCR products (nucleic acid members) are stably associated with a substrate as above and used for gene expression and gene identification studies as described above.

### EXAMPLE III

Identification of a Cytochrome p450 Gene Using a cDNA Microarray Comprising the 3′-End Noncoding Sequences of RNA Transcripts Method of Gene Expression Monitoring

The expression of a cytochrome p450 gene is analyzed by hybridizing target nucleic acids to an array comprising 3′-end noncoding sequences of cytoclnome p45⁰ family members (as described in Example I, above). Samples are isolated or commercially obtained from a biological system, i.e., any of: a cell, a group of cells, a tissue, a group of tissues, an organ, or an organism (e.g., a unicellular or microscopic multicellular organism). Labels are attached to nucleic acids corresponding to RNA transcripts within the sample (“target nucleic acids”) and hybrids between these nucleic acids and the nucleic acid members on the array are detected by detecting the labels.

The incorporation of labels into target nucleic acids is well known in the art and are described hereinabove. A sample comprising labeled target nucleic acids is then contacted with the array under conditions sufficient to allow specific hybridization to occur (e.g., each target labeled transcript molecule hybridizes to its complement and does not hybridize to noncomplementary sequences either in the sample or in the array itself). Suitable hybridization conditions are known in the art and are reviewed in Short Protocols in Molecular Biology, 4^{th} Edition, 1999, ed. Ausubel, et al., the entirety of which is incorporated herein by reference. In one embodiment of the invention, hybridization is performed for 12-24 hours at 42-65° C. in hybridization buffer (e.g., 2× SC). One to a plurality of washes is then performed to remove any unbound molecules or nonspecifically bound molecules from the substrate. In a preferred embodiment of the invention, the array is treated prior to hybridization to minimize nonspecific binding of target molecules. In one embodiment, the array is treated with a solution of 1% “Blotto” or 50 mM tripolyphosphate, or other pre-hybridization solution, routinely used in the art, for at least one hour at 37° C.-50° C. In another embodiment of the invention, blocking nucleic acids are added to the prehybridization solution, e.g., an excess of Alu DNA or polyA oligonucleotides, Cot1 DNA (Human Cot-1 DNA, Life Technologies; Mouse Cot-1 DNA). In still another embodiment of the invention, the array is washed and stripped of bound target molecules (e.g., by boiling in water or 0.5% SDS) to enable reuse of the array.

Detection of hybridization is performed using methods which are appropriate for detecting the label used. In one embodiment, when a colorimetric label is used, hybridization is detected by visualizing the label. In another embodiment, when a radioactive label is used, radiation is detected (e.g., such as by phospho-imaging or autoradiography). In a further embodiment, target nucleic acid molecules are labeled with fluorescent labels and the localization of the label on the array is accomplished by phospho-imaging or by fluorescent microscopy. In one embodiment, the hybridized array is excited with a light source (e.g., a laser) at the excitation wavelength of the particular fluorescent label and the resulting fluorescence at the emission wavelength is detected.

Variations, modifications, and other implementations of what is described herein will occur to those of ordinary skill in the art without departing from the spirit and scope of the invention as claimed. Accordingly, the invention is to be defined not by the preceding illustrative description but instead by the spirit and scope of the following claims. The following references provided include additional information, the entirety of which is incorporated herein by reference.

1. An array comprising a plurality of nucleic acid members, each member having a unique position and stably associated with a solid substrate, wherein each nucleic acid member comprises a sequence of nucleotides corresponding to a non-coding region of a 3′ end of an mRNA transcript, and wherein each of said nucleic acid members is from 10 nucleotides to less than 600 nucleotides.

2. An array comprising a plurality of nucleic acid members, each member having a unique position and stably associated with a solid substrate, wherein each nucleic acid member comprises a sequence of nucleotides corresponding to a non-coding region of a 5′ end of an mRNA transcript, and wherein each of said nucleic acid members is from 10 nucleotides to less than 1000 nucleotides.

3. The array of claim 1 or 2, wherein said noncoding sequence is at least 20 nucleotides in length.

4. The array of claim 1 or 2, wherein each said nucleic acid member comprises substantially noncoding sequences.

5. The array of claim 1, wherein said nucleic acid members comprise human sequences.

6. The array of claim 5, wherein at least one position on said array comprises a control position comprising a substance selected from the group consisting of: a buffer, a cDNA encoded by a housekeeping gene, a plant gene sequence, and a vector sequence.

7. The array of claim 1 or 2, wherein said array comprises from 1000 to 10,000 positions.

8. The array of claim 5, wherein said nucleic acid members comprise sequences expressed in at least two different tissues.

9. The array of claim 1 or 2, wherein said nucleic acid members comprise sequences expressed in a healthy tissue.

10. The array of claim 1 or 2, wherein said nucleic acid members comprise sequences expressed in a diseased tissue.

11. The array of claim 1 or 2, wherein said nucleic acid members comprise sequences expressed in a tissue which has been exposed to a drug.

12. The array of claim 1 or 2 wherein said nucleic acid members do not comprise repeat sequences.
